(19) United States
(12) Patent Application Publication
(10) Pub. No.: US 2017/0014554 A1
(43) Pub. Date: Jan. 19, 2017

(54) SYSTEM AND METHOD FOR ASPIRATING BODILY FLUID

(71) Applicant: H. George Brennan, Newport Beach, CA (US)

(72) Inventor: H. George Brennan, Newport Beach, CA (US)

(21) Appl. No.: 14/799,924

(22) Filed: Jul. 15, 2015

(51) Int. Cl. A61M 1/00; A61M 39/08; A61M 27/00; A61M 39/24

(52) U.S. Cl. CPC A61M 1/0011; A61M 39/24; A61M 39/08; A61M 27/00; A61M 2202/0413; A61M 2202/0014; A61M 2205/7536

(57) ABSTRACT

A medical suction system and method of use is disclosed. The system may include a tube and a collection container. A distal portion of the tube may have a plurality of apertures that are disposed within the patient of an opening adjacent to an incision at a surgical site. A proximal portion of the tube extends out of the opening and is connected to the collection container. The opening is sealed around the tube so as to form a closed system with the collection container. The collection container may be resilient and form a vacuum by compressing by allowing the collection container to decompress. The collection container may also be provided to the user pre-compressed by rolling up the collection container and placing a band or wrapper around the compressed collection container. Alternatively, the collection container may be provided in the uncompressed state. Anchor tabs are used to secure the tube so that the plurality of apertures remain within the patient.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The embodiments disclosed herein relate to a medical suction system and method of use.

When a patient undergoes any invasive surgery, the surgical site will have internal bleeding. After suturing the incision of the surgical site, the fluid from the internal bleeding must be removed. To this end, a tube with a plurality of apertures at the distal portion of the tube is placed within the patient near the surgical site. The tube exits through the skin near the surgical site. The tube provides a conduit through which the bodily fluid from the internal bleeding can flow out of the patient. A vacuum is created oftentimes by use of a resilient drain bulb. The opposite end of the tube is connected to the drain bulb. The drain bulb is manually squeezed to eliminate air from the interior of the bulb and then connected to the opposite end of the tube. Due to the resiliency of the bulb, a vacuum is created which is applied to the tube and aspirates the bodily fluid. The drain bulb may have to be reactivated. Unfortunately, the operation of the drain bulb is unsanitary and typically unsafe. The patient or caregiver (layperson) must disconnect, drain and reactivate the bulb, connect and activate the drain bulb. To do so, a nurse must squeeze the drain bulb right before attaching the drain bulb to the tube. If the patient or caregiver (e.g., nurse) fails to properly install the drain bulb, then the patient's bodily fluids will not be aspirated. Also, if an air leak develops at the incision of the drain site, the drain bulb will expand too rapidly and not aspirate bodily fluid from the surgical site. The exposed portion of the tube may be secured to the person's skin via a roman sandal technique. However, this technique is time consuming for the medical professional to implement. Also, this method does not securely fix the tube to the patient. As such, the tube may be inadvertently pulled out or pushed into the surgical site. If this occurs, the drain site is now exposed to a non-sterile environment. Plus, in order to reestablish the vacuum, surgical intervention is required. The medical professional must surgically open the patient to reposition the tube.

Accordingly, there is a need in the art for an improved device and method for collecting blood or bodily fluids.

### BRIEF SUMMARY

The embodiments disclosed herein address the needs discussed above, discussed below and those that are known in the art.

A medical suction system and method of use are disclosed to provide a securely fixed tube of a drain system and to avoid blood contamination.

The system includes a tube with a plurality of apertures disposed at a distal portion of the tube. The distal portion of the tube may be disposed within the patient's body and extend through an opening closely adjacent to an incision of a surgical site. A proximal portion of the tube extends out of the opening which may be connected to a collection container. The incision at the surgical site may be left open while positioning the distal portion of the tube so that the medical professional can use the incision to visually locate and manipulate the location of the distal portion of the tube. After the tube is in place, the incision at the surgical site is sealed off with a suture or dermabond or other methods known in the art. The opening which receives the tube is formed by a trocar that punctures the skin of the patient. The tube is connected to the back end of the trocar (i.e., needle) so that the tube may be inserted through the opening formed by the trocar similar to a thread on a needle. The tube and the opening form a liquid tight seal, and thus a closed system so that bodily fluid may be drained from the surgical site out of the opening through the tube. The collection container is resilient and is normally biased outward.

The collection container may be pre-compressed so that the user need not simultaneously compress the collection container and connect the proximal portion of the tube to the collection container which requires a heightened manual dexterity not available to all patients or caregivers. The collection container may be provided to the patient or caregiver in a pre-compressed state by rolling up or squeezing the collection container and maintaining the compressed configuration with a band or a wrapper. The band or wrapper may be broken or removed after the proximal portion of the tube is connected to the collection container. The container may have an input port and a one-way valve that prevent spillage of drained bodily fluid in the event that the tube is inadvertently disconnected from the collection container. The benefit of pre-compressing the collection container is that such configuration is easier to use for the patient. The patient or caregiver has less to remember when utilizing the pre-compressed collection container. Moreover, a pre-compressed collection container is smaller in volume therefore less costly to manufacture, ship and store.

More particularly, a bodily fluid container for collecting bodily fluid drained from a patient is disclosed. The container may comprise a resilient body and an input port. The resilient body defines an interior cavity and is provided to a user in a compressed state wherein a volume of the interior cavity is less in the compressed state compared to an uncompressed state. The input port has an input receptacle and a one way valve. The input receptacle is configured to form a liquid tight seal with a first end of a tube. The tube has a second end disposed in the patient to provide fluid communication from the patient to the container. The one way valve allows fluid to flow into and not out of the interior cavity of the container.

The resilient body of the container may be held in the compressed state with a band. Alternatively or additionally, the resilient body may be held in the compressed state with a wrapper.

The input receptacle may be a recess formed in an upper portion of the body having a cylindrical configuration. The recess of the input receptacle may be sized to receive a tube connectable to a patient. The input receptacle may have a cylindrical hole configuration.

The container may further comprise a plug disposed within the input port.

In another aspect, a method of utilizing a container for collecting bodily fluid is disclosed. The method may comprise the steps of providing the container in a compressed state to a user wherein the container is held in the compressed state with a band or a wrapper; activating a fluid flow shut off device to stop flow of fluid through a tube; inserting the tube into an input port of the container; deactivating the fluid flow shut off device to allow flow of fluid through the tube; and removing the band or wrapper from the container to allow the container to expand from the compressed state to an uncompressed state.

The method may further comprise the step of preventing fluid within the container from spilling out of the input port of the container with a one way valve.

The providing step may comprise the step of rolling up the container to compress the container.

The method may further comprise the step of removing a plug from the input port before inserting the tube into the input port.

The method may further comprise the step of activating the fluid flow shut off device to stop flow of fluid through the tube; removing the tube from the input port of the container; inserting the tube into an input port of a second container which is pre-compressed; deactivating the fluid flow shut off device to allow flow of fluid through the tube into the second tube; and removing the band or wrapper from the second container to allow the container to expand from the compressed state to the uncompressed state.

In another aspect, a method of positioning a drainage tube at a surgical site to drain bodily fluid after surgery is disclosed. The method may comprise the steps of attaching a distal end of a tube to a proximal end of a needle; puncturing a skin of the patient to form an opening adjacent to an incision of the surgical site with a sharp distal tip of the needle; pulling the tube through the opening until all apertures formed on a distal portion of the tube is fed through the opening; removing the needle from the tube; disposing the distal portion of the tube under the skin of the patient; and sealing the incision.

The attaching step may comprise the step of inserting the proximal end of the needle into a lumen of the distal end of the tube, an outer diameter of the proximal end of the needle being larger than an inner diameter of the lumen of the tube so that a friction fit engagement forms between the tube and the needle.

The removing step may comprise the step of cutting the tube with a pair of scissors.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 illustrates a surgical site of a patient with a portable, adjustable disposable, medical suction system used to aspirate bodily fluid from the surgical site in a closed system;

FIG. 1A illustrates a trocar attached to a tube for aiding in the formation of a hole in the patient's skin and feeding the tube through that hole;

FIG. 2 illustrates a dead space formed at a surgical site and a trocar directed to an incision;

FIG. 3 illustrates the trocar being inserted into a portion of the skin adjacent to the incision;

FIG. 4 illustrates removal of the trocar from a tube;

FIG. 5 illustrates feeding of the tube until all of the apertures on the tube are disposed on an opposed side of the incision and anchoring of the tube to the skin of the patient;

FIG. 6 illustrates feeding of the tube into the dead space located at the surgical site;

FIG. 7 illustrate placement of the tube in the dead space;

FIG. 8 illustrates a spherical container in a pre-compressed state;

FIG. 9 illustrates a plug of an input port of the spherical container traversed to an opened position so that the input port can accept a tube connected to a cannula for draining bodily fluid into the container;

FIG. 10 illustrates the tube inserted into the input port of the spherical container;

FIG. 11 illustrates bodily fluid partially filling the spherical container;

FIG. 12 illustrates spherical container fully filled with bodily fluid;

FIG. 13 illustrates placement of the tube within the patient at the surgical site; and

FIG. 14 illustrates a container for collecting bodily fluid having an input port and an output port.

### DETAILED DESCRIPTION

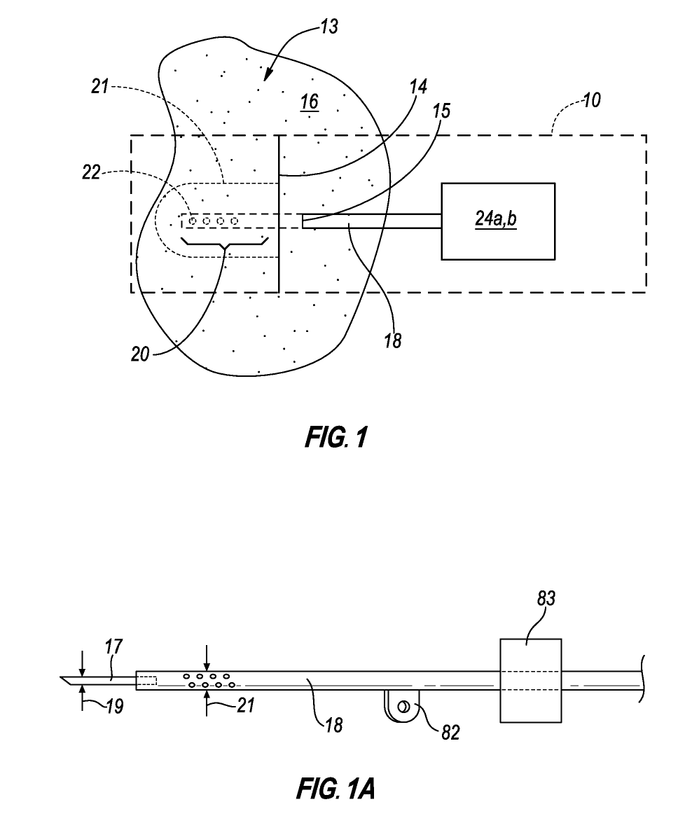

Referring now to the drawings, a portable, adjustable disposable medical suction system 10 (see FIG. 1) and method of use is disclosed. The system 10 is described in relation to implementation for an invasive surgical procedure but may be utilized in other contexts as well. The system 10 may be utilized to aspirate blood/fluid from a patient in a closed system.

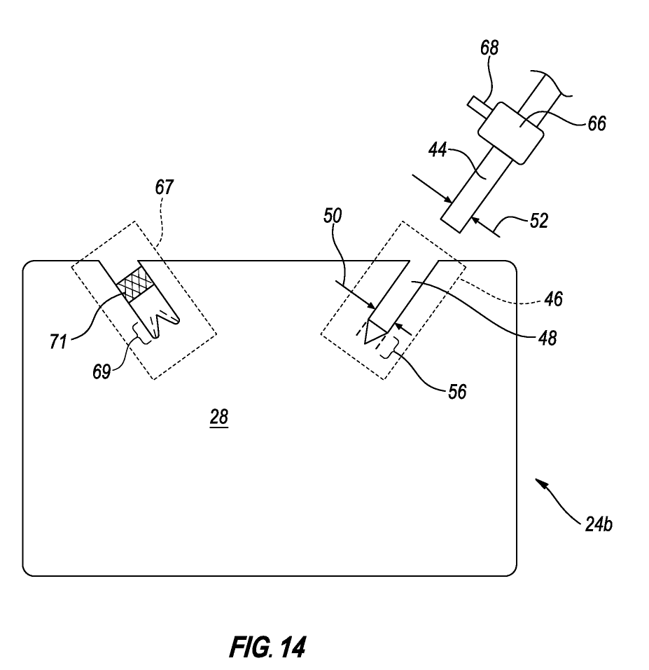

Referring to FIG. 1, the portable, adjustable disposable medical suction system 10 may be utilized to withdraw blood or bodily fluid from internal bleeding caused by surgery or trauma as shown in FIG. 1. In particular, the surgical site 13 of a patient 12 may have an incision 14 through a skin 16 of the patient 12. An opening 15 may be formed immediately adjacent to the incision 14. An elongate tube 18 may be disposed in the opening 15 adjacent to the incision 14 of the surgical site and form a liquid tight seal therewith. A distal portion 20 of the elongate tube 18 may have a plurality of apertures 22. The distal portion 20 is disposed in a dead space 21 under the skin 16 of the patient 12 through the opening 15 at the surgical site 13. The incision may be closed with a dermabond, suturing or other techniques known in the art or developed in the future. The distal portion 20 of the tube 18 extends into the dead space 21 under the skin of the patient 12, while a proximal portion of the tube 18 extends out of the body of the patient 12. The proximal portion of the tube 18 which extends out of the body of the patient 12 is connected to a collection container 24a, b. The collection container 24a, b is resilient and may be compressed before attachment of the collection container 24a, b to the tube 18 so as to form a vacuum in the collection container 24a, b as it 24a, b is being decompressed. The collection container 24a may be pre-compressed and held in the pre-compressed state until use by way of a band or wrapper. Alternatively, the collection container 24b (see FIG. 14) may be uncompressed which requires the user to compress the container 24b in order to prime the system so that the container 24b can create a vacuum to aspirate bodily fluid from the patient. After the collection container 24a, b is connected to the tube 18, the band or wrapper is broken or removed from the pre-compressed container 24a to allow the collection container 24a to expand and form the vacuum therein. If the container 24b was not pre-compressed, then the user needs to compress the container 24b in order to create the vacuum. The vacuum created by the decompression of the collection container 24a, b draws blood/fluid out of the body of the patient 12 through the apertures 22, through the tube 18 and into the collection container 24a, b. The collection container 24a, b is removably attachable to the tube 18. The collection container 24a, b may be sealable (e.g., one-way valve and/or plug) so that after removal from the tube 18 when the collection container is full, the bodily fluid (e.g., blood, moisture, etc.) can be stored in the collection container until it can be safely discarded at the hospital, outpatient care center or otherwise safely discarded. The collection container full of bodily fluid may also be brought back to the doctor for inspection with respect to the volume of fluid collected in the collection container and potential infections that may be observed in the collected fluid in the collection container.

Additional collection containers 24a, b may be sequentially attached to the tube 18 as more blood is aspirated and if more blood or bodily fluid needs to be aspirated from the surgical site.

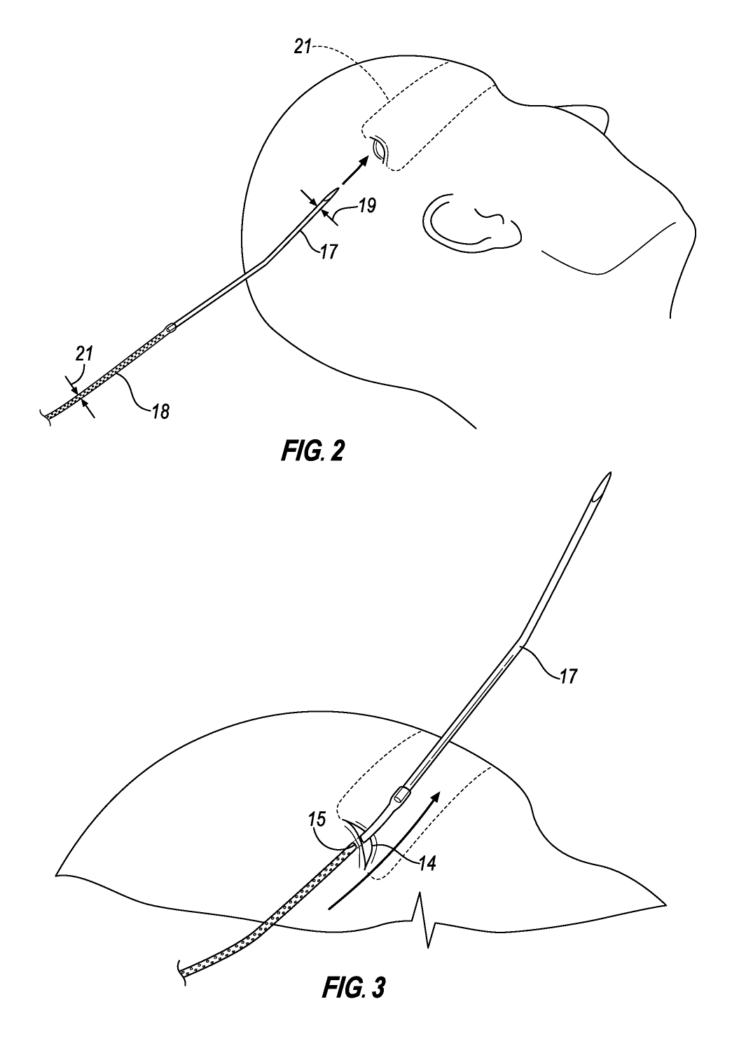

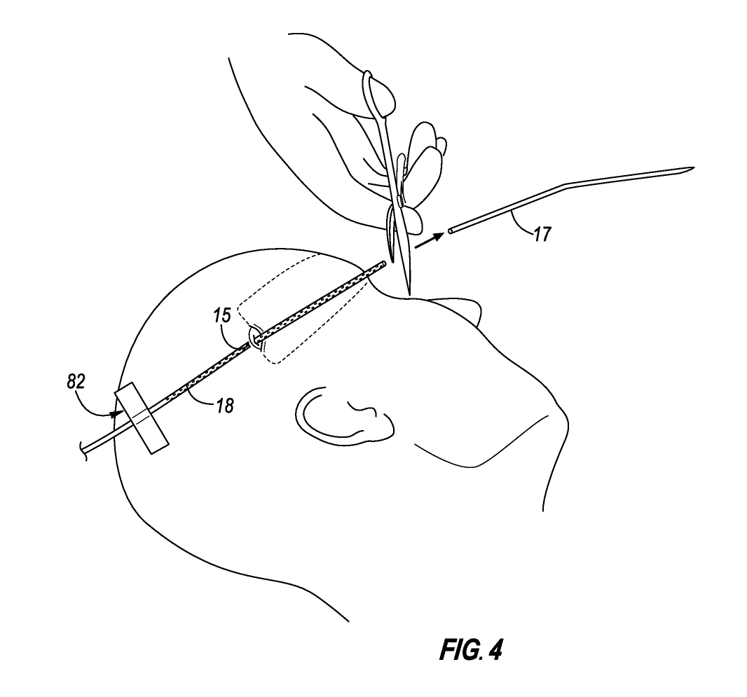

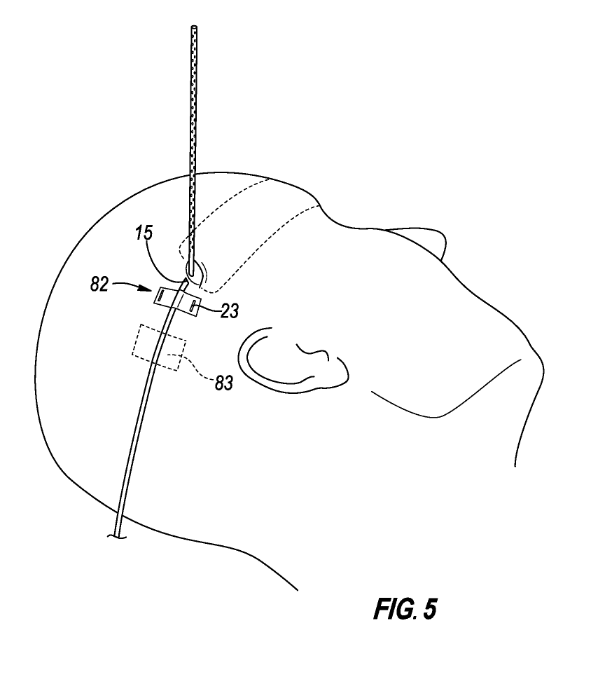

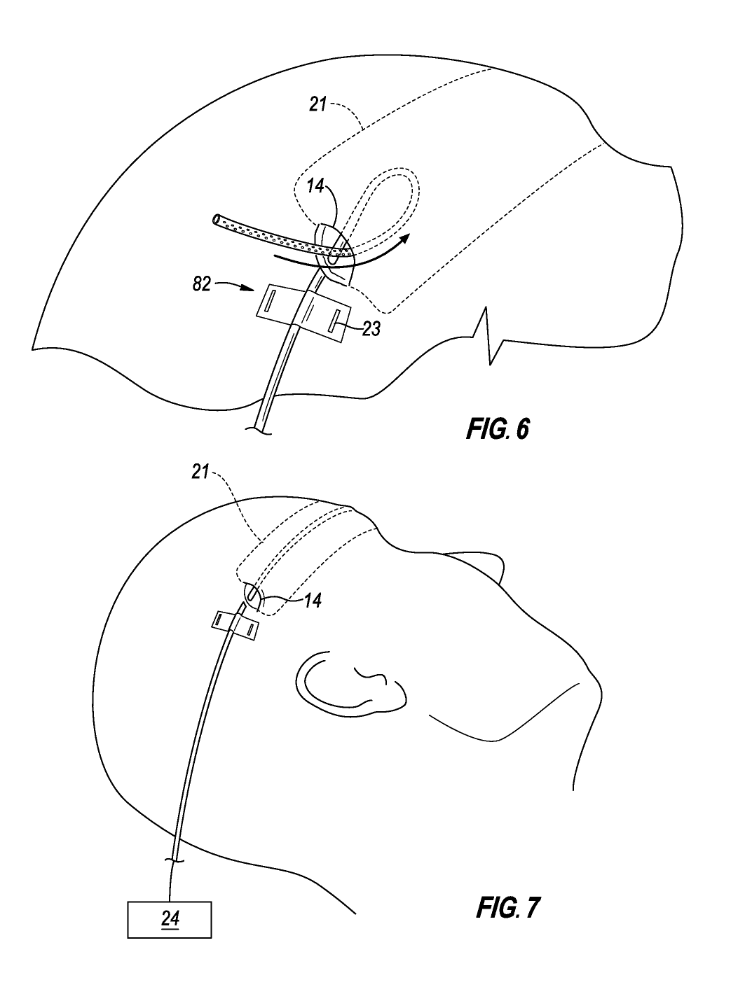

The collection container 24a, b is disposable, and thus sanitary for the patient and medical professional. The collection container 24a, b may also be brought back to the doctor for inspection and analysis. An example of a closed air system is illustrated in FIG. 1. After invasive surgery, a surgical site will have internal bleeding. The medical professional may insert the tube 18 into an opening 15 of the skin of the patient adjacent to the incision 14 as shown in FIGS. 2-7. The incision 14 may be large enough to aid the medical professional in visually locating the distal portion of the tube. The tube 18 may be inserted into the opening 15 with the aid of a trocar or a cannula inserter 17. (see FIGS. 1A and 2). The trocar 17 may have an outer diameter 19 which is equal to an outer diameter 21 of the tube 18. The tube 18 is slipped over the end of the trocar 17 so as to form a friction hold therewith. A distal tip of the trocar 17 has a sharp tip and is used to puncture the skin of the patient adjacent to the incision 14 to form the opening 15. The trocar 17 is pulled through the opening 15. Since the outer diameter 19 of the trocar 17 is equal to (or less than) the outer diameter 21 of the tube 18, the size of the opening 15 is perfectly sized to the outer diameter of the tube 18. The opening 15 of the skin, being elastic, bears down on the outer surface of the tube 18 to form the liquid tight seal therewith. The opening 15 is formed near the incision 14. The trocar 17 is directed toward the incision 14, as shown in FIGS. 2 and 3, so that the medical professional may view the tube 18 through the incision 14 to place the tube 18 in its proper position. The skin at the opening 15 stretches around the tube 18 and forms the liquid tight seal therebetween. Referring now to FIG. 4, once the tube 18 is disposed through the opening 15, the trocar 17 may be removed from the tube 18. In this regard, the tube 18 may be fabricated typically from a plastic material that is cut with a pair of scissors. The distal portion of the tube 18 has a plurality of apertures that extends between the distal end and an anchor system 82. The anchor system 82 may be any embodiment disclosed in U.S. Ser. No. 13/892,131, filed on May 10, 2014, the entire contents of which is expressly incorporated herein by reference. Additionally, the anchor system 82 may include a tape 83 that adheres the tube 18 to the patient's skin. If the tape 83 needs to be applied to the patient's skin where hair exists, then the hair may be shaven off before application of the tape 83 to the area. Alternatively, the anchor system 82 for securing the tube to the skin of the patient may be an anchor tab or hooks and loops. The tube 18 is slid into the opening 15 until all of the apertures are disposed on the other side of the opening 15, as shown in FIG. 5. The tube 18 is pulled through the opening 15 until the anchor system 82 is disposed closely adjacent to the opening 15. The anchor system 82 may be secured or fixed to the patient's skin with staples 23 or suture. By way of example and not limitation, the anchor system 82 may be staples, sutures, tape. The tape 83 adheres the tube 18 to the patient's skin which is shaven if required since tape 83 does not adhere to hair to prevent movement of the tube. The distal portion of the tube 18 may be fed under the patient's skin, as shown in FIG. 6 and placed at the surgical site as shown in FIG. 7. More particularly, a dead space 21 is created under the patient's skin which detaches the patient's skin from the subcutaneous tissue. The dead space 21 is formed before the tube 18 is introduced adjacent to the surgical site with the trocar 17. As shown in FIG. 2, the dead space 21 has already been formed. The incision 14 may be closed by suturing or with a dermabond. The proximal portion of the tube 18 is connected to the collection container 24a or 24b. Here, the collection container 24a, b generates a vacuum so as to draw only bodily fluids from the inside of the patient into the collection container 24a, b. The collection container 24a, b does not require a power generated vacuum mechanism such as a pump. Rather, the collection container 24a, b generates a vacuum due to the resiliency of the wall of the collection container 24a, b. In particular, if the collection container 24a, b is pre-compressed or compressed then released, the resiliency of the wall of the collection container 24a, b expands the collection container 24a, b. The expansion generates a vacuum which draws in the bodily fluid from the inside of the patient.

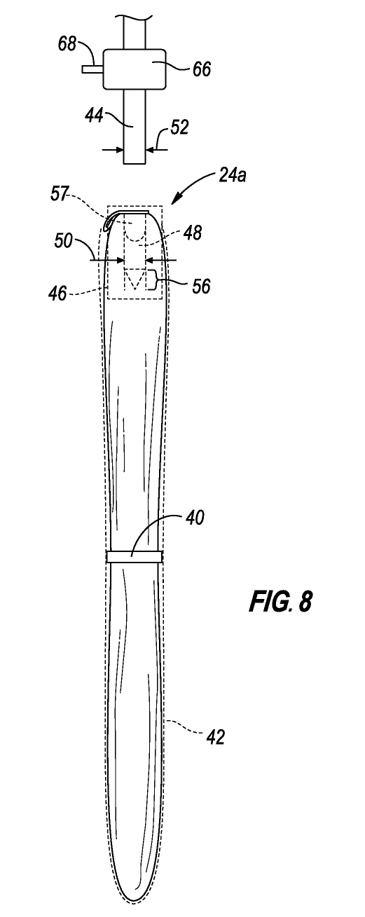

More particularly, referring now to FIGS. 8-12 and 14, the container 24a, b may be fabricated from a resilient material that is bendable and flexible. The container 24a, b is formed so that in its natural state, the walls of the container 24a, b are expanded outward and define an interior cavity 28. The volume of the interior cavity 28 may be about three (3) fluid ounces. The container 24a may be provided to the medical professional in a pre-compressed state. Alternatively, the container 24b may be provided to the medical professional in an uncompressed state (see FIG. 14). When the pre-compressed container 24a is released or the uncompressed container 24b is compressed then released, the walls of the container 24a, b expand outward to its normally outward biased state. The container 24a, b may have a spherical or other bolus configuration as shown in FIG. 4. Other configurations are also contemplated such as rectangular as shown in FIG. 14. The container 24a may be provided to a medical professional or patient in the compressed state as shown in FIG. 8. In particular, the container 24a may be rolled up with a band 40 wrapped around the rolled up container 24a. Alternatively or additionally, the rolled up container 24a may be wrapped in the pre-compressed state with a breakable wrapper 42 which encompasses the rolled up container 24a. The wrapper 42 does not cover the plug 57 so that the plug 57 can be traversed to the opened position with the wrapper 42 disposed about the pre-compressed container 24a. By pre-compressing the collection container 24a, the system is easier to use by the patient because the patient need not be required to compress the collection container 24a before attaching the collection container 24a to the tube disposed within the patient. Additionally, during shipping, the pre-compressed collection container 24a is smaller in volume therefore less costly to manufacture, ship and store. Upon breakage of the band 40 or wrapper, the resiliency of the walls of the container 24a urges the container 24a to its original expanded shape. The expansion of the walls of the container 24a creates a vacuum within the interior cavity 28. Alternatively, if the container 24b is provided to the medical professional or patient in the uncompressed state as shown in FIG. 14, the user needs to compress the collection container 24b before attaching the collection container 24b to the tube 18 disposed within the patient. The walls of the container 24a may be fabricated from an elastomeric material, a plastic material, or any other material that is resilient and causes the walls of the container 24 to expand outward.

FIG. 1 illustrates use of the container 24a, b in relation to a closed air system. In particular, the container 24a, b is utilized to ensure all bodily fluid from the patient is drawn out of the patient's body and urged into the container 24a, b. In one embodiment, the container 24a may be provided pre-compressed and held in that position with the band 40 or wrapper. By pre-compressing the container 24a, the system is easier to use by the patient since the patient need not remember to compress the container 24a before attaching the container 24a to the tube disposed within the patient. Moreover, the pre-compressed container 24a is smaller in volume, and thus less costly to manufacture, ship and store. To create a vacuum within the container 24a, the person breaks off or removes the band 40 or wrapper from the rolled up container 24a. If the container 24a is in fluid communication to the wound, then the vacuum within the interior cavity 28 of the container 24a is applied to a tube 44 that connects the container 24a to the wound. Alternatively, in another embodiment, the container 24b may be provided in an uncompressed state.

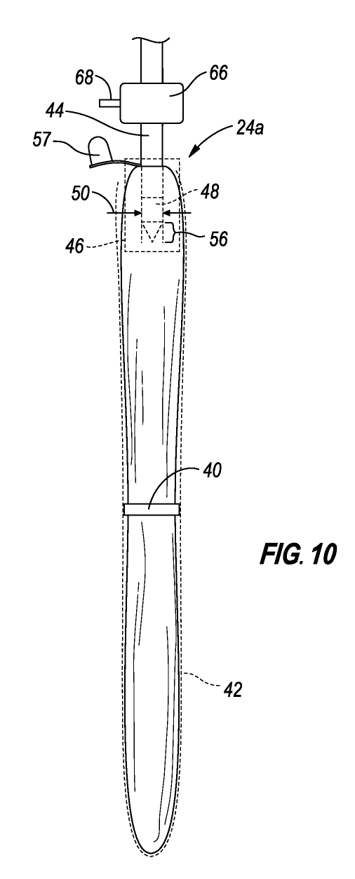

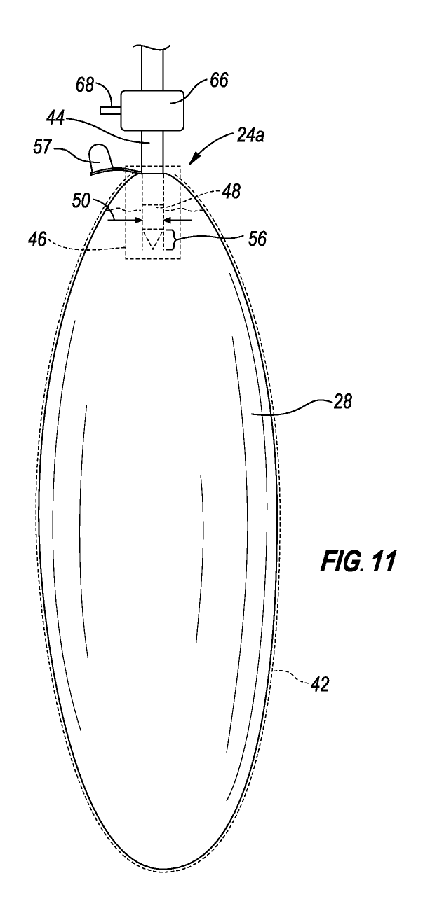

Referring to FIGS. 8 and 14, container 24a, b may have an input port 46. The input port 46 may have a receptacle 48. This receptacle 48 is sized and configured to form an airtight seal with the tube 44 which is in fluid communication with the wound of the patient. By way of example and not limitation, the receptacle 48 may be formed as a cylindrical hole. A diameter 50 of the cylindrical hole defining the receptacle 48 may be slightly smaller than a diameter 52 of the tube 44. In this manner, when the tube 44 is inserted into the receptacle 48 (see FIG. 10), the exterior surface of the tube 44 contacts and provides a tight fit with the internal surface of the receptacle 48. Although the receptacle 48 is shown and described as a cylindrical hole, other shapes for the receptacle 48 are also contemplated. By way of example and not limitation, the receptacle 48 may have a funnel shape, rectangular shape, oval-shape or any other shape that achieves an airtight seal with the tube 44. Additionally, the receptacle 48 may be retrofitted with a connector that can receive the tube 44 and form an airtight and liquid tight seal therewith.

The input port 46 may additionally have a one-way valve 56. The one-way valve 56 allows fluid to be transferred from outside of the container 24a, b into the cavity 28. However, the one-way valve 56 at the input port 46 prevents fluid from being transferred from inside of the container 24a, b to the outside of the container 24a, b when the tube is disconnected from the input port 46. In the example shown, the one-way valve 56 allows bodily fluid to be transferred from the patient or person to the internal cavity 28. When the tube 44 is removed from the receptacle 48, the one-way valve 56 prevents the bodily fluid contained within the container 24a, b from flowing out of the input port 46.

The tube 44 which is connected to the input port 46 may additionally have a pinch valve 66. The pinch valve 66 allows the person to shut off fluid flow through the tube 44. When the pinch valve 66 is on, no fluid passes through the tube 44. When the pinch valve 66 is off, fluid can freely pass through the tube 44. The pinch valve 66 may be operated by actuating a lever 68 to turn the pinch valve 66 on or off. By way of example and not limitation, the pinch valve 66 may operate so as to have a shut off mechanism disposed within the tube 44. To turn the pinch valve 66 on or off, the mechanism disposed within the tube 44 may be rotated or shifted 90 degrees. This may be accomplished by means known in the art or developed in the future such as through mechanical linkages or magnetism. In another example, the pinch valve 66 may be a simple clamp that compresses the tube 44 by actuation of a handle.

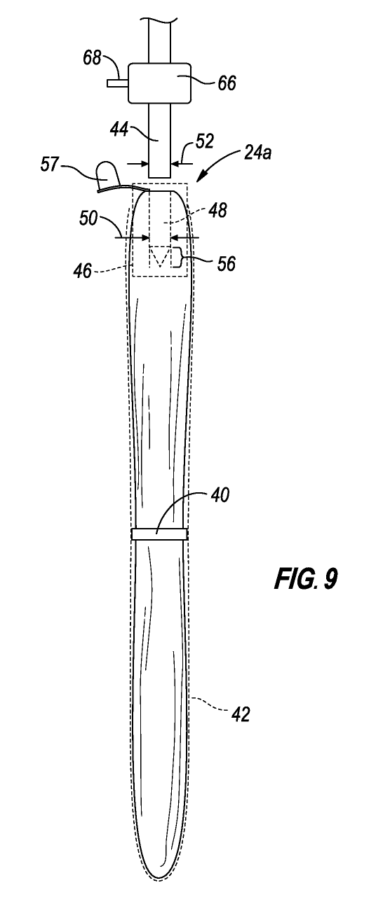

To utilize the container 24a, b in a closed system such as when draining bodily fluid from an invasive surgical procedure, the container 24a may initially be provided to the medical professional or user in a pre-compressed state, as shown in FIG. 8. Alternatively, the container 24b may be provided to the medical professional or user in an uncompressed state, as shown in FIG. 14. In the decompressed state, the internal cavity of the collection container 24a, b may hold about three (3) fluid ounces. If the collection container 24a is provided in the pre-compressed state, the person need not squeeze the container 24a to reduce the size of the internal cavity 28 before connecting the container 24a to the tube 44 disposed within the patient, as discussed below. Alternatively, if the collection container 24b is provided in the uncompressed state, the person needs to squeeze the container 24b to reduce the size of the internal cavity 28 before connecting the container 24b to the tube 44 disposed within the patient, as discussed herein. With the pinch valve 66 on so that no fluid passes through the tube 44 and the pre-compressed container 24a in its compressed state or the uncompressed container 24b held in the compressed state by the user's hand, the plug 57 is removed from the input port 46, as shown in FIG. 9. Thereafter, the distal end of a proximal portion of the tube 44 is inserted into the receptacle 48, as shown in FIG. 10. The tight fit between the receptacle 48 and the tube 44 forms an airtight or liquid tight seal therebetween. Moreover, the connection between the tube 44 and the receptacle 48 is easy and does not require complex motor skills. Also, when changing out the collection container 24a, b for an empty new collection container 24a, b, there is minimal risk of blood contamination since the pinch valve 66 prevents spillage of blood from within the tube 44 and the one way valve 56 of the collection container 24a, b prevents spillage of any blood from the collection container 24a, b. For the pre-compressed container 24a, when the band 40 is removed from the rolled up container 24a, gas is not allowed to flow through the tube 44 since the pinch valve 66 is turned on. A vacuum is formed in the interior cavity of the container 24a. The container 24a remains compressed even though the band 40 is removed from the rolled up container 24a.

The other end (i.e., distal portion) of the tube 44 is disposed within a dead space 21 under the skin of the body of the patient through the opening 15 adjacent to the incision 14 at the wound site to drain any bodily fluid that might be produced because of the wound. The proximal portion of the tube 44 disposed within the patient's body may have a plurality of holes 22 as shown in FIG. 1. The opening 15 of the patient's skin that receives the tube 44 is sealed off so that air does not enter the holes 22 and destroy the vacuum. The tube 44 is inserted into the skin with the use of a cannula inserter 17. The cannula inserter 17 punctures the skin and forms the opening 15. In this way, the patient's skin is wrapped tightly around the tube 44 and forms a liquid tight seal therewith. Hence, the container 24a, b is being utilized in a closed system.

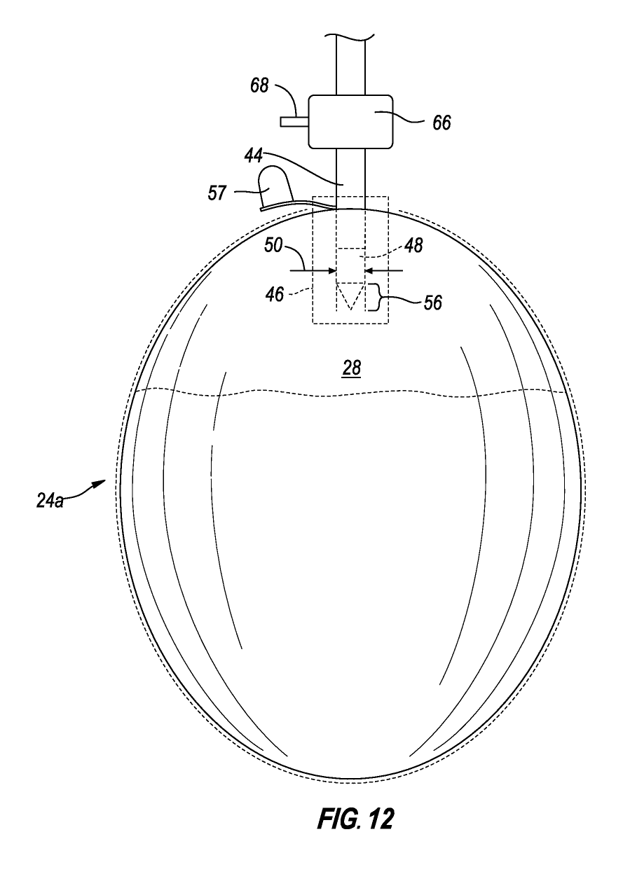

The pinch valve 66 is now turned off so that fluid can flow through the tube 44. The vacuum created by the compressed container 24a, b draws bodily fluid from the wound into the internal cavity 28 of the container 24a, b. As the container 24a, b expands out, as shown in FIG. 11, the bodily fluid is drawn into the container 24a, b. As the container 24a, b continues to expand outward, the vacuum pressure created by the container 24a, b decreases. At a certain point in time, the vacuum created by the container 24a, b is insufficient to draw bodily fluid from the patient or the container 24a, b is fully expanded outward and no vacuum is created, as shown in FIG. 12.

When the vacuum created by the container 24a, b is absent or insufficient to draw bodily fluid from the wound of the patient into the container 24a, b, the container 24a, b may be changed out with a new container 24a, b in the following manner. The pinch valve 66 is turned on by actuating lever 68. At this point, no fluid is allowed to pass through the tube 44. The tube 44 is removed from the receptacle 48. The one-way valve 56 of the input port 46 prevents the bodily fluid within the container 24a, b from leaking out of the container 24a, b. Moreover, the filter 62 at the output port 58 prevents the bodily fluid from leaking out of the container 24b through the output port 58. The used container 24a, b may be placed in a red bag and sealed for disposal. In contrast, if the container 24a, b is not pre-compressed, then the caregiver or patient must remember to first compress the container 24b before connecting the container to the tube 44.

If a pre-compressed container 24a is used, the new pre-compressed container 24a may now be connected to the tube 44. The person can easily and quickly connect the tube 44 into the receptacle 48 of the input port 46 without needing complex motor skills to do so. The collection container 24a may have a plug in the input port to prevent debris from entering the collection container prior to use. The plug 57 may first be removed while the collection container 24a of FIG. 5 is still in the pre-compressed state with the band 40 or wrapper 42. Moreover, there is minimal or no risk of blood contamination. The one way valve 56 of the old collection container 24a prevents spillage of blood and the pinch valve 66 prevents blood from spilling out of the tube 44. A tight fit between the tube 44 and receptacle 48 creates a liquid and airtight seal therebetween. When the band 40 is removed from the rolled up container 24a, a vacuum is created within the container 24a. The pinch valve 66 is now activated to the off position to allow fluid to flow through the tube 44. Bodily fluid then continues to flow into the container 24a until desired or until the body no longer produces bodily fluid. The container 24a may be replaced as desired or prescribed by a medical professional. If an uncompressed container 24b is used, then the same procedures for replacing the container 24a may be utilized for replacing the container 24a with an uncompressed container 24b. Moreover, the container 24b may be compressed either before or after connecting the tube 44 in the receptacle 48. To compress the container 24b, the user grips the container 24b and squeezes the container 24b with his or her hand. Air within the container 28 escapes out of an output port 67 that has a one-way valve 69 that allows air to escape out of the output port 67 but does not allow air to enter the container 28. The output port 67 additionally may have a membrane 69 that allows air to pass through the membrane but not liquid such as the hydrophobic filter.

As discussed herein, the holes 22 formed in the tube 44 are disposed within the body of the patient. Sometimes, accidentally, the hole 22 closest to the wound opening slips out and is exposed to the atmosphere. By way of example and not limitation, after the tube 44 is disposed within the patient's body, the patient may move around to eat, use the restroom, drive, etc. While moving around, the tube 44 may be pulled out by accident and expose the proximal hole 22 to the atmosphere. If the vacuum is lost in the container 24a, b, because the cannula or tube 44 slips out so that air would simply reinflate the container 24a, b, surgical intervention is required which would be undesirable. As such, it is preferable that the tube 44 does not slip so that the holes 22 are not exposed to the atmosphere. To this end, preferably, the anchor system 82 is utilized to secure the tube 44 in a stationary position.

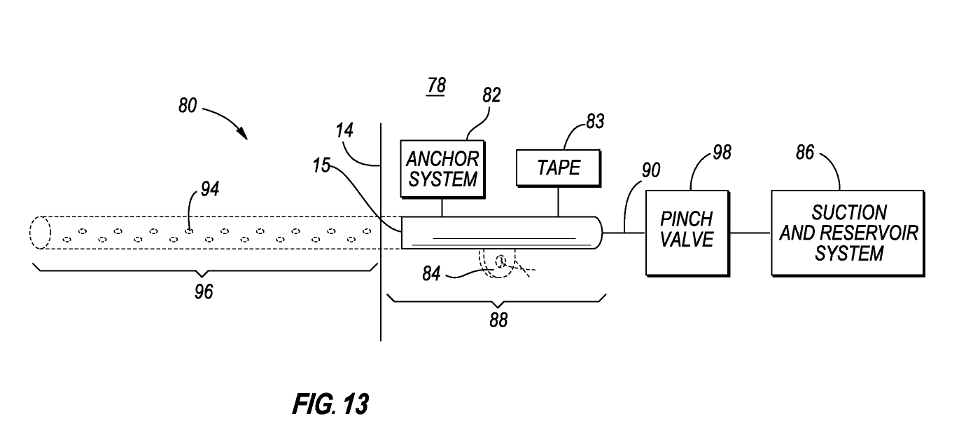

Referring now to FIG. 13, the tube may be a drainage cannula 80. The tube 18 or the cannula 80 may be anchored to a skin 78 of a patient is shown. The tube 18 or drainage cannula 80 may be anchored to the skin of the patient with any of the various embodiments and aspects of an anchor system 82 described in U.S. patent application Ser. No. 13/892,131, filed on May 10, 2014, the entire content of which is expressly incorporated herein by reference. By way of example and not limitation, the anchor system 82 includes and is not limited to the tabs 84 and hooks and loops described in U.S. patent application Ser. No. 13/892,131. In addition to the anchor system 82 described in U.S. patent application Ser. No. 13/892,131, the anchor system 82 may also include a tape 83 for adhering the cannula or tube 44 to the skin of the patient. The anchor system 82 prevents slippage of the tube 44 or drainage cannula so that the tube holes or holes of the drainage cannula do not exit the body cavity of the patient. If the drainage cannula or tube holes are exposed to the atmosphere, then air would simply re-inflate the collection container 24a, b and defeat the purposes of the system. As such, it is important to secure the tube 44 in its proper place. If the tube 44 slips, then it may be necessary to reinsert the tube during surgery which would be an undesirable result.

In FIG. 13, the drainage cannula 80 is shown. However the various aspects discussed in relation to FIG. 13 may be also applied to the tube 18 discussed in relation to FIGS. 1-12. The cannula 80 may be fluidically attached to a suction and reservoir system 86. In particular, a proximal portion 88 of the cannula 80 may have an opening which is connected to a tube 90. The tube 90 may be connected to suction container 24a, b shown in the figures and describe herein. The drainage cannula 80 may be inserted into the body opening 15 of the patient adjacent to the incision 14. The suction container 24a, b may be provided to the user pre-compressed as described in relation to FIGS. 8-12. Alternatively, the suction container 24b may be provided to the user uncompressed as described in relation to FIG. 14. After the tube from the drainage cannula 80 is connected to the suction container, the band 40 or wrapper 42 around the suction container may be removed or broken to allow the resiliency of the material of the suction container 24a to expand the suction container 24a and create a vacuum which draws the bodily fluid from the patient's body into the suction container 24a.

The drainage cannula 80 may have a plurality of apertures 94 formed on the distal portion 96 thereof. Also, a pinch valve 98 may be in line between the drainage cannula 80 and the suction and reservoir system 86.

Any fluid in the container 24a, b may be saved and brought back to the doctor for inspection and analysis. By way of example and not limitation, the doctor may check for volume of fluid being drained as well as any infections that may be detected visually or through subsequent tests of the collected bodily fluid.

The above description is given by way of example, and not limitation. Given the above disclosure, one skilled in the art could devise variations that are within the scope and spirit of the invention disclosed herein, including various ways of connecting the first and second lines to the vacuum source 18. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A bodily fluid container for collecting bodily fluid drained from a patient, the container comprising:
a resilient body defining an interior cavity and provided to a user in a compressed state wherein a volume of the interior cavity is less in the compressed state compared to an uncompressed state;
an input port having:
an input receptacle configured to form a liquid tight seal with a first end of a tube, the tube has a second end disposed on the patient to provide fluid communication from the patient to the container;
a one way valve for allowing fluid to flow into and not out of the interior cavity.

2. The container of claim 1 wherein the resilient body is held in the compressed state with a band.

3. The container of claim 1 wherein the resilient body is held in the compressed state with a wrapper.

4. The container of claim 1 wherein the input receptacle is a recess formed in an upper portion of the body having a cylindrical configuration, and the recess of the input receptacle is sized to receive a tube connectable to a patient.

5. The container of claim 1 wherein the input receptacle has a cylindrical hole configuration.

6. The container of claim 1 further comprising a plug disposed within the input port.

7. The container of claim 1 further comprising an output port for allowing air but not fluid for escaping out of the interior cavity of the container, the output port having:
a one way valve oriented so that fluid can escape out of the interior cavity of the container;
a hydrophobic filter for allowing air but not bodily fluid to pass therethrough.

8. A method of utilizing a container for collecting bodily fluid, the method comprising the steps of:
providing the container in a compressed state to a user wherein the container is held in the compressed state with a band or a wrapper;
activating a fluid flow shut off device to stop flow of fluid through a tube;
inserting the tube into an input port of the container;
deactivating the fluid flow shut off device to allow flow of fluid through the tube;
removing the band or wrapper from the container to allow the container to expand from the compressed state to an uncompressed state.

9. The method of claim 8 further comprising the step of preventing fluid within the container from spilling out of the input port of the container with a one way valve.

10. The method of claim 9 wherein the providing step comprises the step of rolling up the container to compress the container.

11. The method of claim 9 further comprising the step of removing a plug from the input port before inserting the tube into the input port.

12. The method of claim 9 further comprising the step of:
activating the fluid flow shut off device to stop flow of fluid through the tube;
removing the tube from the input port of the container;
inserting the tube into an input port of a second container which is precompressed;
deactivating the fluid flow shut off device to allow flow of fluid through the tube into the second tube;
removing the band or wrapper from the second container to allow the container to expand from the compressed state to the uncompressed state.

13. A method of positioning a drainage tube at a surgical site to drain bodily fluid after surgery, the method comprising the steps of:
attaching a distal end of a tube to a proximal end of a needle;
puncturing a skin of the patient to form an opening adjacent to an incision of the surgical site with a sharp distal tip of the needle;
pulling the tube through the opening until all apertures formed on a distal portion of the tube is fed through the opening;
removing the needle from the tube;
disposing the distal portion of the tube under the skin of the patient;
sealing the incision.

14. The method of claim 13 wherein the attaching step comprises the step of inserting the proximal end of the needle into a lumen of the distal end of the tube, an outer diameter of the proximal end of the needle being larger than an inner diameter of the lumen of the tube so that a friction fit engagement forms between the tube and the needle.

15. The method of claim 13 wherein the removing step comprises the step of cutting the tube with a pair of scissors.
